(19) United States
(12) Patent Application Publication
(10) Pub. No.: US 2020/0330336 A1
(43) Pub. Date: Oct. 22, 2020

(54) NON-MEDICATED FEMALE LUBRICATING AND MOISTURIZING GEL

(71) Applicants: MARIE JARDINES, Miami, FL (US); Ruben Alcoba, Miami Springs, FL (US)

(72) Inventors: MARIE JARDINES, Miami, FL (US); Ruben Alcoba, Miami Springs, FL (US)

(21) Appl. No.: 16/391,292

(22) Filed: Apr. 22, 2019

(51) Int. Cl. A61K 8/04; A61K 8/92; A61K 8/67; A61K 8/9789; A61K 9/00; A61K 8/34

(52) U.S. Cl. CPC A61K 8/042; A61K 8/922; A61K 8/678; A61K 8/345; A61K 8/671; A61K 8/9789; A61K 9/0034; A61K 8/675

(57) ABSTRACT

A non-medicated female lubricating and moisturizing gel that is used by women to lubricate and moisturize their vagina. The gel will increase the pleasure of a woman whom suffers from vaginal dryness by providing a lubricant that will increase the sensitivity of the cutaneous areas while soothing the areas. The gel is composed of a distilled H2O, at least one antioxidant, at least one thickening agent, at least one preservative, at least one emulsifier, at least one skin penetrator, a soothing agent, a glycerin, and at least one nitric oxide generating natural supplement.

### BACKGROUND

The present invention is directed to a non-medicated female lubricating and moisturizing gel that is used by women to lubricate and moisturize their vagina.

Vaginal dryness is affects women of all ages and it is particularly common during and after menopause.¹ Women whom suffer from vaginal dryness normally feel discomfort during intercourse. ¹ Available at https://www.ncbi.nim.nih.gov/pmc/articles/PMC4819835/, Edwards D, Panay N. Treating vulvovaginal atrophy/genitourinary syndrome of menopause: how important is vaginal lubricant and moisturizer composition?. Climacteric. 2015;19(2):151-161. doi:10.3109/13697137.2015.1124259, seen on Apr. 21, 2019.

The impact of vaginal dryness on interpersonal relationships, quality of life, daily activities, and sexual function can be significant to the woman afflicted with vaginal dryness.² ² Id.

It is estimated that approximately 15% of premenopausal and up to 57% of postmenopausal women experience vaginal dryness.³ ³ Id.

Lack of natural lubrication is one of the more commonly encountered sexual problems in the clinical care setting.⁴ Women report that they prefer vaginal-penile intercourse to feel wetter, for they feel that they are more easily orgasmic when the sex is wetter, and they believe that their partners prefer sex to feel wetter rather than dryer.⁵ It stand to reason that women whom suffer from vaginal dryness will become more fertile when they use a lubricant or moisturizer. ⁴ Id.⁵ Id.

Using a lubricant will reduce the pain and discomfort felt by a woman whom suffers from vaginal dryness. Reducing the pain felt by the woman during intercourse will allow the woman to relax and possibly lead to the woman becoming more fertile.

The present invention provides a lubricant that will moisturize the vaginal areas of a woman with compounds that will increase the wetness of the vaginal areas while providing nutrients that will increase the blood flow and the sensitivity of the vaginal areas while also soothing the vaginal areas with a soothing agent. Increasing the blood flow of the vaginal areas will allow a woman using the gel to feel more sensitivity when being penetrated and will increase her pleasure. The soothing agent will relieve some of the pain normally associated with intercourse and in turn increase the pleasure felt during intercourse. The increase in pleasure may lead to the woman to be more fertile and orgasmic.

For the foregoing reason there is a need for a non-medicated female lubricating and moisturizing gel that is used by women to lubricate and moisturize their vagina, that will reduce the pain associated with intercourse, and increase the sensitivity of the vaginal areas.

### SUMMARY

The present invention describes a non-medicated female lubricating and moisturizing gel that is used by women to lubricate and moisturize their vagina. The gel will increase the pleasure of a woman whom suffers from vaginal dryness by providing a lubricant that will increase the sensitivity of the cutaneous areas while soothing the areas.

The non-medicated female lubricating and moisturizing gel of the present invention is composed of a distilled H2O, at least one antioxidant, at least one thickening agent, at least one preservative, at least one emulsifier, at least one skin penetrator, a soothing agent, a glycerin, and at least one nitric oxide generating natural supplement.

The soothing agent of the present invention also serves to increase the cutaneous blood flow. The above statements is being made based on the known research that states, “it is . . . accepted that a sympathetic cholinergic active vasodilator system is responsible for the sizeable increase in skin blood flow during hyperthermia.”⁶ Menthol is a compound that will trigger the skin to increase blood flow circulation and thereby increase the sensitive of the areas in which it has been placed on. Menthol works by triggering the nerve endings of cells to believe that it is cool outside, mimicking hyperthermia.⁷ ⁶ Available at https://www.ncbi.nim.nih.gov/pmc/articles/PMC5356216/, Wong B J, Hollowed C G. Current concepts of active vasodilation in human skin. Temperature (Austin). 2016;4(1):41-59. Published 2016 Jun. 21. doi:10.1080/23328940.2016.1200203, seen on Apr. 21, 2019.⁷ Available at https://www.scientificamerican.com/article/how-we-sense-the-heat-of-chili-peppers-and-the-cool-of-menthol-excerpt/, Scientific American, How We Sense the Heat of Chili Peppers and the Cool of Menthol, by David J. Linden, seen on Apr. 21, 2019.

The higher sensitivity in the skin immersed with the gel of the present invention will allow the person using the gel to experience greater pleasure while minimizing the normal pain associated vaginal dryness.

The skin penetrators of the present composition serve to allow the nitric oxidant generating supplements to penetrate the dermis of the vaginal areas and help vasodilate the veins and arteries under the dermis to increase blood flow. It is believed that the increase of blood flow to the vaginal areas will enhance the sensations felt during intercourse. The skin penetrators while also allow anti-oxidants to penetrate the skin to repair any damage that may have been caused by intercourse.

The present composition uses vitamins to provide antioxidants to the vaginal areas to promote regeneration of cells damaged during intercourse or by age.

Glycerin is used in the present composition in order to provide a safe lubricant that will cause minimal harm if ingested. Although not recommended, it also serves to give the composition a sweet taste that will enhance the experience of individuals practicing oral sex.

The present composition, in some embodiments, also uses scents to increase the sexual arousal of those whom use the cream and those whom may be partners of those using the cream. Vanilla is a natural aphrodisiac the simultaneously induces euphoria and relaxation and it is used in an embodiment of the present composition.⁸ It is believed that the scent of a cinnamon oil extract will increase penile blood flow⁹. ⁸ Available at https://thoughtcatalog.com/lorenzo-jensen-iii/2015/07/17-naturally-erotic-scents-that -will-drive-your-partner-or-c-r-a-z-y-in-bed/, 17 Naturally Erotic Scents That Will Drive Your Partner C-R-A-Z-Y In Bed, by Lorenzo Jensen III, Jul. 11 2015, seen on April 21, 2019.⁹ Id.

An object of the gel of the present invention is to provide women with a gel that will lubricate their vaginal areas.

Another object of the present invention is to provide women with a gel that will increase vaginal sensitivity that may lead to female fertility.

Yet another object of the present invention is to provide a gel that will increase the blood flow of the vaginal areas.

Still another object of the present invention is to provide a gel that will rejuvenate the dermis of the vaginal areas.

Yet still another object of the present invention is to provide a soothing gel that can be used by women during intercourse.

Yet still another object of the present invention is to provide a gel that will work as an aphrodisiac.

### DESCRIPTION

The present invention is a non-medicated female lubricating and moisturizing gel. In an embodiment of the present invention, the gel comprises of a distilled H2O, at least one antioxidant, at least one thickening agent, at least one preservative, at least one emulsifier, at least one skin penetrator, a soothing agent, a glycerin, and at least one nitric oxide generating natural supplement.

The at least one antioxidant is selected from the group consisting of a Vitamin B3, a Vitamin E, or a Vitamin A. The Vitamin B3 is in the range from about 0.0095 oz to about 0.0104 oz, the Vitamin E is in the range from about 0.0025 oz to about 0.0035 oz, and the Vitamin A is in the range from about 0.0015 oz to about 0.0025 oz. Vitamin B3 and Vitamin E are placed in the gel to reduce the collagen degradation of the dermis of the vaginal areas of a woman. Vitamin A in the gel is used to promote the metabolism of collagen in the vaginal areas. The vitamins of the gel are placed in the gel to promote the rejuvenation of the dermis of the vaginal areas.

The at least one thickening agent is a Carbomer. The Carbomer is in the range from about 0.0035 oz to about 0.0045 oz.

The at least one preservative is selected from the group consisting of a Sodium Benzoate, a Sodium Sorbate, a SALIGUARD PG or a Caprylic/Capric Triglyceride. The Sodium Benzoate is in the range from about 0.0025 oz to about 0.0035 oz, the Sodium Sorbate is in the range from 0.0025 oz to about 0.0035 oz, the SALIGUARD PG is in the range from about 0.0025 oz to about 0.0035 oz, and the Caprylic/Capric Triglyceride is in the range from about 0.0025 oz to about 0.0035 oz.

The at least one emulsifier is selected from the group consisting of a Propylene Glycol or a Triethanolamine. The Propylene Glycol is in the range from about 0.0095 oz to about 0.0105 oz and the Triethanolamine is in the range from about 0.0025 oz to about 0.0035 oz.

The at least one skin penetrator is selected from the group consisting of a D-Limonene or an Aloe Vera. The D-Limonene is in the range from about 0.0035 oz to about 0.0045 oz and the Aloe Vera is in the range from about 0.0005 oz to about 0.0065 oz. The skin penetrators are used in the gel to aid the anti-oxidants and the nitric oxide geniting natural supplements to reach the veins and arteries of the vaginal areas so that they can vasodilate and therefore allow blood to more freely pass through the veins and arteries.

The soothing agent is a menthol. The menthol is in the range from about 0.0001 oz to about 0.0003 oz. The soothing agent, when applied to the dermis of the vaginal areas, mimics a cold sensation on the dermis of the skin and the cold sensation triggers greater blood flow in the areas.

The at least one nitric oxide generating natural supplement is selected from the group consisting of a CoQ10, an L-Arginine, an L-Citrulline or a watermelon seed oil. The CoQ10 is in the range from about 0.0001 oz to about 0.0003 oz, the L-Arginine is in the range from about 0.0035 oz to 0.0045 oz, and the watermelon seed oil is in the range from about 0.0035 oz to about 0.0045 oz. The nitric oxide generating natural supplements aid in the vasodilation of the veins and arteries of the vaginal areas so that greater blood flow passes through the veins and arteries. It is believed that greater blood flow through the veins and arteries will increase the sensitivity of the vaginal areas and will also promote rejuvenation of the dermis of the vaginal areas.

And, the distilled H2O is in the range from about 0.9055 oz to about 0.9298 oz and the Glycerin is in the range from about 0.0150 oz to about 0.0250 oz.

In another embodiment of the present invention, the gel comprises of a Black Maca powder.

The Black Maca powder is in the range from about 0.0045 oz to about 0.0055 oz. Black Maca is believed to increase energy and fertility in mammals.¹⁰ ¹⁰ Available at https://www.ncbi.nim.nih.gov/pmc/articles/PMC3184420/, Gonzales G F. Ethnobiology and Ethnopharmacology of Lepidium meyenii (Maca), a Plant from the Peruvian Highlands. Evid Based Complement Alternat Med. 2011;2012:193496. doi:10.1155/2012/193496, seen on April 22, 2019.

In still another embodiment of the present invention, the gel comprises of a vanilla oil and a cinnamon oil. The vanilla oil is in the range from about 0.0025 oz to about 0.0035 oz and the cinnamon oil is in the range from about 0.0001 oz to about 0.0010 oz. The vanilla oil and the cinnamon oil are placed in the gel so that the gel will give of an aphrodisiac scent that will promote intimacy between two partners.

In yet still another embodiment of the present invention, the gel comprises of a Pomegranate seed oil, a Safflower oil, and a Cannabidiol oil. The Pomegranate seed oil is in the range from about 0.0025 oz to about 0.0035 oz, the Safflower oil is in the range from about 0.0025 oz to about 0.0035 oz, and the cannabidiol oil is in the range from about 0.0025 oz to about 0.0035 oz.

An advantage of the gel of the present invention is that it provides women with a gel that lubricates their vaginal areas.

Another advantage of the present invention is that it provides women with a gel that increases vaginal sensitivity.

Yet another advantage of the present invention is that it provides a gel that increases the blood flow of the vaginal areas.

Still another advantage of the present invention is that it provides a gel that rejuvenates the dermis of the vaginal areas.

Yet still another advantage of the present invention is that it provides a soothing gel that can reduce pain during intercourse.

Yet still another advantage of the present invention is that it provides a gel that works as an aphrodisiac.

While the inventor's above description contains many specificities, these should not be construed as limitations on the scope, but rather as an exemplification of several preferred embodiments thereof. Accordingly, the scope should not be determined by the embodiments illustrated, but by the appended claims and their legal equivalents.

1. A non-medicated female lubricating and moisturizing gel, the gel comprises:
a distilled H2O;
at least one antioxidant;
at least one thickening agent;
at least one preservative;
at least one emulsifier;
at least one skin penetrator;
a soothing agent;
a glycerin; and
at least one nitric oxide generating natural supplement.

2. The non-medicated female lubricating and moisturizing gel of claim 1, wherein:
the at least one antioxidant is selected from the group consisting of a Vitamin B3, a Vitamin E, or a Vitamin A;
the at least one thickening agent is a Carbomer;
the at least one preservative is selected from the group consisting of a Sodium Benzoate, a Sodium Sorbate, a Phenoxyethanol or a Caprylic/Capric Triglyceride;
the at least one emulsifier is selected from the group consisting of a Propylene Glycol or a Triethanolamine;
the at least one skin penetrator is selected from the group consisting of a D Limonene or an Aloe Vera;
the soothing agent is a menthol; and
the at least one nitric oxide generating natural supplement is selected from the group consisting of a CoQ10, an L-Arginine, an L-Citrulline or a watermelon seed oil.

3. The non-medicated female lubricating and moisturizing gel of claim 2, wherein every oz of the gel has in weight:
the distilled H2O is in the range from about 0.9055 oz to about 0.9298 oz;
the Vitamin B3 is in the range from about 0.0095 oz to about 0.0104 oz, the Vitamin E is in the range from about 0.0025 oz to about 0.0035 oz, and the Vitamin A is in the range from about 0.0015 oz to about 0.0025 oz;
the Carbomer is in the range from about 0.0035 oz to about 0.0045 oz;
the Sodium Benzoate is in the range from about 0.0025 oz to about 0.0035 oz, the Sodium Sorbate is in the range from 0.0025 oz to about 0.0035 oz, the Phenoxyethanol is in the range from about 0.0025 oz to about 0.0035 oz, and the Caprylic/Capric Triglyceride is in the range from about 0.0025 oz to about 0.0035 oz;
the Propylene Glycol is in the range from about 0.0095 oz to about 0.0105 oz and the Triethanolamine is in the range from about 0.0025 oz to about 0.0035 oz;
the D-limonene is in the range from about 0.0035 oz to about 0.0045 oz and the Aloe Vera is in the range from about 0.0005 oz to about 0.0065 oz;
the menthol is in the range from about 0.0001 oz to about 0.0003 oz;
the Glycerin is in the range from about 0.0150 oz to about 0.0250 oz; and
the CoQ10 is in the range from about 0.0001 oz to about 0.0003 oz, the L-arginine is in the range from about 0.0035 oz to 0.0045 oz, and watermelon seed oil is in the range from about 0.0035 oz to about 0.0045 oz.

4. The non-medicated female lubricating and moisturizing gel of claim 3, the gel comprises a Black Maca powder.

5. The non-medicated female lubricating and moisturizing gel of claim 4, wherein the Black Maca powder is in the range from about 0.0045 oz to about 0.0055 oz.

6. The non-medicated female lubricating and moisturizing gel of claim 5, the gel comprises:
a vanilla oil; and
a cinnamon oil.

7. The non-medicated female lubricating and moisturizing gel of claim 6, wherein the vanilla oil is in the range from about 0.0025 oz to about 0.0035 oz and the cinnamon oil is in the range from about 0.0001 oz to about 0.0010 oz.

8. The non-medicated female lubricating and moisturizing gel of claim 7, the gel comprises:
a Pomegranate seed oil;
a Safflower oil; and
a Cannabidiol oil.

9. The non-medicated female lubricating and moisturizing gel of claim 8, wherein the Pomegranate seed oil is in the range from about 0.0025 oz to about 0.0035 oz, the Safflower oil is in the range from about 0.0025 oz to about 0.0035 oz, and the cannabidiol oil is in the range from about 0.0025 oz to about 0.0035 oz.
